(19) United States
(12) Patent Application Publication
(10) Pub. No.: US 2003/0073591 A1
(43) Pub. Date: Apr. 17, 2003

(54) Effervescent detergent composition

(76) Inventors: Kevin Todd Norwood, Cincinnati, OH (US); Charles David Bragg, Woolsington (GB)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 09/933,020

(22) Filed: Aug. 20, 2001

(51) Int. Cl. A61K 7/30; C11D 17/00

(52) U.S. Cl. 510/117; 510/447; 510/446; 510/509

(57) ABSTRACT

Detergent compositions having a detersive surfactant system, an effervescent system comprising an acid source comprising an acid or acid-salt blend having the formula

ZO₂C—(CR₅R₆)ₓ—C—R₁—R₂—M—C—R₃—R₄—(CR₇R₈)_{y}—CO₂Z;

wherein xis 0 or 1, y is 0 or 1,

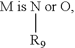

R₁, R₂, R₃, R₄, R₅, R₆, R₇, R₈ and R₉ are independently selected from the group consisting of H, OH, branched or linear alkyl hydrocarbon, branched or linear alkenyl hydrocarbon, branched or linear alcohol, branched or linear sulfonate or sulfate salt, branched or linear amine, branched or linear ether, branched or linear carboxylic acid, carboxylate salt substituents having a total of 6 or less total carbon units and mixtures thereof and wherein Z is selected from the group consisting of an alkali metal and hydrogen, with the proviso that on average greater than 1 Z, as measured in the complete acid or acid-salt blend, is hydrogen; and an alkali source are combined such that the acid source and the alkali source are capable of reacting together in the presence of water to form a gas.

### FIELD OF THE INVENTION

This invention relates to effervescent detergent compositions and the use of specific organic builders as the acid source in an effervescent detergent composition. More particularly, the present invention relates to granular detergent compositions intended for the cleaning of fabrics comprising the specific organic builders disclosed herein.

### BACKGROUND OF THE INVENTION

In order for a granular detergent composition to be effective in cleaning it is necessary that it dissolve adequately in the wash solution. Generally, when diluted with water the tendency of typical granular detergents is toward poor dissolution. That tendency has been exacerbated by the recent trend in the detergent industry towards higher bulk density granular compositions and towards granular detergent compositions, which have a higher content of active ingredients. Granular detergent compositions of high bulk densities ranging from 650 to 1100 kg/m3 are attractive to consumers but can sometimes be difficult to satisfactorily dissolve in an aqueous medium.

Another difficulty with high density or compact detergent compositions is that they are not easily flushed from the dispenser drawer of a washing machine. Similar problems are encountered when using such granular detergent compositions in a dosing device in the washing drum.

The dissolution problems associated with granular detergent compositions are further exacerbated when the composition is formed into a tablet. Whether the tablet is formed using pressure compaction or simply lightly compacted and coated, tablets tend to be more difficult to dissolve in the wash water, largely because of the decreased surface area initially exposed to the water.

Previous attempts in the art to combat low dissolution incorporate citric acid and a carbonate source in the detergent compositions to promote dissolution via effervescence. This is especially true in pharmaceutical preparations. In order to ensure a uniform distribution of these effervescent materials in these preparations it is essential to use sodium bicarbonate and citric acid in the form of fine powders. It is also necessary to incorporate considerable proportions of these effervescent materials in the preparations in order to obtain the desired effect. Furthermore, a major issue with such powder compositions is the poor storage stability when they are exposed to moisture.

In recent years there has been much interest in developing effervescent detergent products. The inclusion of effervescence in a detergent product aids in dissolution of the detergent in the wash water as well as adds a consumer perceived efficacy signal. Current attempts at delivering effervescent detergent compositions include the use of systems comprising citric acid and a carbonate source. The citric acid and carbonate react in the presence of water to produce carbon dioxide bubbles and therefore effervescence.

Most laundry detergents today require large amounts of ingredients that sequester metal ions in the wash water, commonly referred to as builders, to improve the cleaning performance of the detergents. Some of the drawbacks related to the use of effervescent systems employing citric acid as the acid source are the fact that citric acid has a relatively low calcium sequestration constant and thus is a poor builder in addition to being expensive. Therefore, these systems are not ideal for commercial purposes.

Accordingly the need remains for an effervescent system for incorporation into a detergent composition, which provides superior dissolution. The need also remains for an effervescent system that provides builder benefits in addition to improved dissolution and is economical to use.

### SUMMARY OF THE INVENTION

The present invention solves the aforementioned needs by providing an effervescent detergent composition comprising, a detersive surfactant system, and an effervescent system comprising, an acid source comprising an acid or acid-salt blend having the formula

ZO₂C—(CR₅R₆)ₓ—C—R₁—R₂—M—C—R₃—R₄—(CR₇R₈)_{y}—CO₂Z; (I)

wherein x is 0 or 1, y is 0 or 1,

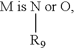

R₁, R₂, R₃, R₄, R₅, R₆, R₇, R₈ and R₉ are independently selected from the group consisting of H, OH, branched or linear alkyl hydrocarbon, branched or linear alkenyl hydrocarbon, branched or linear alcohol, branched or linear sulfonate or sulfate salt, branched or linear amine, branched or linear ether, branched or linear carboxylic acid, carboxylate salt substituents having a total of 6 or less total carbon units and mixtures thereof and wherein Z is selected from the group consisting of an alkali metal and hydrogen, with the proviso that on average greater than 1 Z constituent, as measured in the complete acid or acid-salt blend, is hydrogen, wherein the acid source and the alkali source are capable of reacting together in the presence of water to form a gas.

It is thus an object of the present invention to provide compositions with improved dissolution and/or dispensing characteristics upon dilution in an aqueous medium. It is a further object of the present invention to provide compositions with a multifunctional acid source, providing both improved dissolution and builder capacity.

All percentages, ratios, and proportions herein are on a weight basis unless otherwise indicated. All documents cited are hereby incorporated by reference in their entirety.

### DETAILED DESCRIPTION OF THE INVENTION

This invention provides an effervescent detergent composition comprising a detersive surfactant system and an effervescent system as well as conventional detergent ingredients. The effervescent system is comprised of an acid source and an alkali source capable of reacting together in the presence of water to form a gas.

Effervescent System

The effervescent system of the present invention comprises an acid source and an alkali source. The effervescent system contains from about 20% to about 80% by weight, of an acid source and from about 80% to about 20% by weight of an alkali source. Generally, detergent compositions contain from about 5% to about 50% by weight of the total composition of the effervescent system, preferably from about 7% to about 30% and more preferably from about 10% to about 20%. It is also preferred that the ratio of acid source to alkali source be in the range of from about 1:2 to about 2:1.

Acid Source

The acid source preferably has the formula I as detailed below.

ZO₂C—(CR₅R₆)ₓ—C—R₁—R₂—M—C—R₃—R₄—(CR₇R₈)_{y}—CO₂Z; (I)

wherein x is 0 or 1, y is 0 or 1,

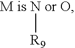

R₁, R₂, R₃, R₄, R₅, R₆, R₇, R₈ and R₉ are independently selected from the group consisting of H, OH, branched or linear alkyl hydrocarbon, branched or linear alkenyl hydrocarbon, branched or linear alcohol, branched or linear sulfonate or sulfate salt, branched or linear amine, branched or linear ether, branched or linear carboxylic acid, carboxylate salt substituents having a total of 6 or less total carbon units and mixtures thereof and wherein Z is selected from the group consisting of an alkali metal (selected from the group consisting of sodium and potassium) and hydrogen, with the proviso that on average greater than 1 Z constituent, as measured in the complete acid or acid-salt blend, is hydrogen;

The acid source must have sufficient acidity to drive the effervescent reaction. In general this means the pKa of at least one of the acid groups associated with the acid source according to the present invention is less than or equal to the pKa of carbonic acid. Compounds of the general formulas described above can be used in the present invention in the acid form alone or as a partially neutralized acid/salt form. Use of the completely deprotonated salt form exclusively will result in no effervescence and is not within the scope of this invention. However, one of ordinary skill will recognize that mixture of completely deprotonated and partially neutralized or acidic materials are well within the scope.

Preferred acid sources include but are not limited to:

nitrilotriacetic acid (NTA) or mixed acid/salt blends corresponding to formula I described above wherein x=0, y=0, M═N—R₉, R₁, R₂, R₃, and R₄ are all H and R₉ is CH₂CO₂Z;

methyglycine diacetic acid (MGDA) or mixed acid/salt blends corresponding to formula I described above wherein x=0, y=0, M═N—R₉, R₁, R₂, R₃, and R₄ are all H and R₉ is CH(CH₃)CO₂Z; L-iminodisuccinic acid (LIDS) or mixed acid/salt blends corresponding to corresponding to formula I described above wherein x=1, y=1, M═N—R₉, R₁ and R₃ are H, R₂ and R₄ are CO₂Z, R₅, R₆, R₇, R₈ and R₉ are H; L-hydroxyiminodisuccinic acid (LHIDS) or mixed acid/salt blends corresponding to formula I described above wherein x=1, y=1, M═N—R₉, R₁ and R₃ are H, R₂ and R₄ are CO₂Z, R₅, R₆, R₇, and R₉ are H and R₈ is OH; carboxymethyl oxysuccinic acid (CMOS) or mixed acid/salt blends corresponding to formula I described above wherein x=0, y=1, M═O, R₁, R₂R₃R₇, and R₈ are H, and R₄ are CO₂Z;

oxydisuccinic acid (ODS) or mixed acid/salt blends corresponding to corresponding to formula I described above wherein x=1, y=1, M═O, R₁, R₃R₅, R₆, R₇ and R₈ are H, and R₂R₄ are CO₂Z.

In accordance with the invention the acid source, which is capable of reacting with the alkali source to produce a gas, is preferably capable of building and/or binding free ions such as calcium and magnesium ions, present in the wash.

Alkali Source

In accordance with the present invention, an alkali source is present such that it has the capacity to react with the source of acid dispersing aid to produce a gas. Preferably this gas is carbon dioxide, and therefore the alkali is a carbonate, or a suitable derivative thereof.

The detergent composition of the present invention preferably contains from about 2% to about 75%, preferably from about 5% to about 60%, most preferably from about 10% to about 35% by weight of the alkali source. When the alkali source is present in an agglomerated detergent particle, the agglomerate preferably contains from about 10% to about 60% of the alkali source.

In a preferred embodiment, the alkali source is a carbonate. Examples of preferred carbonates are the alkaline earth and alkali metal carbonates, including sodium carbonate, bicarbonate and sesqui-carbonate and any mixtures thereof with ultra-fine calcium carbonate such as are disclosed in German Patent Application No. 2,321,001 published on Nov. 15, 1973. Alkali metal percarbonate salts are also suitable sources of carbonate species and are described in more detail in the section ‘inorganic perhydrate salts’ herein.

The alkalinity system may include, in addition to the alkali sources described above, other components, such as silicate. Suitable silicates include the water soluble sodium silicates with an SiO₂:Na₂O ratio of from 1.0 to 2.8, with ratios of from 1.6 to 2.0 being preferred, and 2.0 ratio being most preferred. The silicates may be in the form of either the anhydrous salt or a hydrated salt. Alkali metal persilicates are also suitable sources of silicate herein. Preferably the silicates have an amorphous structure.

Other suitable sources will be known to those skilled in the art.

Optional Detersive Ingredients

As a preferred embodiment, the conventional detergent ingredients are selected from typical detergent composition components such as detersive surfactants and detersive builders. Optionally, the detergent ingredients can include one or more other detersive adjuncts or other materials for assisting or enhancing cleaning performance, treatment of the substrate to be cleaned, or to modify the aesthetics of the detergent composition. Usual detersive adjuncts of detergent compositions include the ingredients set forth in U.S. Pat. No. 3,936,537, Baskerville et al. and in Great Britain Patent No. 2,311,296, Trinh et al., granted Dec. 29, 1999. Such adjuncts are included in detergent compositions at their conventional art-established levels of use, generally from 0% to about 80% of the detergent ingredients, preferably from about 0.5% to about 20% and can include color speckles, suds boosters, suds suppressors, antitarnish and/or anticorrosion agents, soil-suspending agents, soil release agents, dyes, fillers, optical brighteners, germicides, alkalinity sources, hydrotropes, antioxidants, enzymes, enzyme stabilizing agents, solvents, solubilizing agents, chelating agents, clay soil removal/anti-redeposition agents, polymeric dispersing agents, processing aids, fabric softening components, static control agents, bleaching agents, bleaching activators, bleach stabilizers, etc.

Surfactants

The hand and/or machine washing detergent compositions of the present invention may optionally comprise a non mid-chain branched alkyl sulfate or non-mid chain branched aryl sulphonate surfactant. Depending upon the embodiment of the present invention one or more categories of surfactants may be chosen by the formulator. Preferred categories of surfactants are selected from the group consisting of anionic, cationic, nonionic, zwitterionic, ampholytic surfactants, and mixtures thereof. Within each category of surfactant, more than one type of surfactant of surfactant can be selected. Surfactant is preferably present in an amount of from about 0.1% to about 99%, preferably from about 5% to about 50%, more preferably from about 10% to about 20% by weight of the composition.

Nonlimiting examples of surfactants useful herein include:

a) C₁₁-C₁₈ alkyl benzene sulfonates (LAS);

b) C₁₀-C₂₀ primary, branched-chain and random alkyl sulfates (AS);

C) C₁₀-C₁₈ secondary (2,3) alkyl sulfates having the formula:

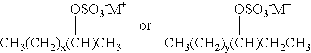

wherein x and (y+1) are integers of at least about 7, preferably at least about 9; said surfactants disclosed in U.S. Pat. No. 3,234,258 Morris, issued Feb. 8, 1966; U.S. Pat. No. 5,075,041 Lutz, issued Dec. 24, 1991; U.S. Pat. No. 5,349,101 Lutz et al., issued Sep. 20, 1994; and U.S. Pat. No. 5,389,277 Prieto, issued Feb. 14, 1995 each incorporated herein by reference;

d) C₁₀-C₁₈ alkyl alkoxy sulfates (AE_{X}S) wherein preferably x is from 1-7;

e) C₁₀-C₁₈ alkyl alkoxy carboxylates preferably comprising 1-5 ethoxy units;

f) C₁₂-C₁₈ alkyl ethoxylates, C₆-C₁₂ alkyl phenol alkoxylates wherein the alkoxylate units are a mixture of ethyleneoxy and propyleneoxy units, C₁₂-C₁₈ alcohol and C₆-C₁₂ alkyl phenol condensates with ethylene oxide/propylene oxide block polymers inter alia Pluronic® ex BASF which are disclosed in U.S. Pat. No. 3,929,678 Laughlin et al., issued Dec. 30, 1975, incorporated herein by reference;

g) Alkylpolysaccharides as disclosed in U.S. Pat. No. 4,565,647 Llenado, issued Jan. 26, 1986, incorporated herein by reference;

h) Polyhydroxy fatty acid amides having the formula:

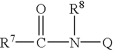

wherein R⁷ is C₅-C₃₁ alkyl; R⁸ is selected from the group consisting of hydrogen, C₁-C₄ alkyl, C₁-C₄ hydroxyalkyl, Q is a polyhydroxyalkyl moiety having a linear alkyl chain with at least 3 hydroxyls directly connected to the chain, or an alkoxylated derivative thereof; preferred alkoxy is ethoxy or propoxy, and mixtures thereof; preferred Q is derived from a reducing sugar in a reductive amination reaction, more preferably Q is a glycityl moiety; Q is more preferably selected from the group consisting of —CH₂(CHOH)ₙCH₂OH, —CH(CH₂OH)(CHOH)ₙ₋₁CH₂OH, —CH₂(CHOH)₂—(CHOR′)(CHOH)CH₂OH, and alkoxylated derivatives thereof, wherein n is an integer from 3 to 5, inclusive, and R′ is hydrogen or a cyclic or aliphatic monosaccharide, which are described in U.S. Pat. No. 5,489,393 Connor et al., issued Feb. 6, 1996; and U.S. Pat. No. 5,45,982 Murch et al., issued Oct. 3, 1995, both incorporated herein by reference.

Additionally and preferably, the surfactant may be a midchain branched alkyl sulfate, midchain branched alkyl alkoxylate, or midchain branched alkyl alkoxylate sulfate. These surfactants are further described in PCT Application No. WO 99/19434, published Apr. 22, 1999, PCT Application No. WO 99/18929, published Apr. 22, 1999, U.S. Pat. No. 6,228,829 issued May 8, 2001, U.S. application Ser. No. 09/542,684 allowed Apr. 10, 2001, PCT Application No. WO 99/19448 published Apr. 22, 1999 and U.S. application Ser. No. 09/543,087 allowed Apr. 10, 2001. Other suitable mid-chain branched surfactants can be found in U.S. Pat. No. 6,008,181, issued Dec. 28, 1999, U.S. Pat. No. 6,060,443, issued May 9, 2000, U.S. Pat. No. 6,020,303, issued Feb. 1, 2000, U.S. Pat. No. 6,093,856, issued Jul. 25, 2000, PCT Application No. WO 97/38972 published Oct. 23, 1997, U.S. Pat. No. 6,046,152, issued Apr. 4, 2000, and U.S. Pat. No. 6,015,781, issued Jan. 18, 2000. Mixtures of these branched surfactants with conventional linear surfactants are also suitable for use in the present compositions.

Other preferred anionic surfactants are the modified alkyl benzene sulfonate surfactants, or MLAS. Some suitable MLAS surfactants, methods of making them and exemplary compositions are further described in U.S. Pat. No. 6,274,540, issued Aug. 14, 2001, PCT Application No. WO 99/05242, published Feb. 4, 1999, U.S. application Ser. No. 09/479,365, allowed Jun. 7, 2001, PCT Application No. WO 99/05082, published Feb. 4, 1999, PCT Application No. WO 99/05084, published Feb. 4, 1999, PCT Application No. WO 99/05241, published Feb. 4, 1999, PCT Application No. WO 99/07656, published Feb. 18, 1999, PCT Application No. WO 00/23549, published Apr. 27, 2000, and PCT Application No. WO 00/23548, published Apr. 27, 2000.

Detergency Builders

The detergent composition may also include a conventional detergent builder to assist in controlling mineral hardness and to enhance the removal of particulate soils. Inorganic or P-containing detergent builders include, but are not limited to, the alkali metal, ammonium and alkanolammonium salts of polyphosphates (exemplified by the tripolyphosphates, pyrophosphates, and glassy polymeric meta-phosphates), phosphonates, phytic acid, silicates, carbonates (including bicarbonates and sesquicarbonates), sulphates, and aluminosilicates. However, non-phosphate builders are required in some locations. Importantly, the compositions herein function surprisingly well even in the presence of the so-called “weak” builders (as compared with phosphates) such as citrate, or in the so-called “underbuilt” situation that may occur with zeolite or layered silicate builders.

Examples of silicate builders are the alkali metal silicates, particularly those having a SiO₂:Na₂O ratio in the range 1.6:1 to 3.2:1 and layered silicates, such as the layered sodium silicates described in U.S. Pat. No. 4,664,839, issued May 12, 1987 to H. P. Rieck. NaSKS-6 is the trademark for a crystalline layered silicate marketed by Hoechst (commonly abbreviated herein as “SKS-6”). Unlike zeolite builders, the Na SKS-6 silicate builder does not contain aluminum. NaSKS-6 has the delta-Na₂SiO₅ morphology form of layered silicate. It can be prepared by methods such as those described in German DE-A-3,417,649 and DE-A-3,742,043. SKS-6 is a highly preferred layered silicate for use herein, but other such layered silicates, such as those having the general formula NaMSiₓO₂ₓ₊₁·yH₂O wherein M is sodium or hydrogen, x is a number from 1.9 to 4, preferably 2, and y is a number from 0 to 20, preferably 0 can be used herein. Various other layered silicates from Hoechst include NaSKS-5, NaSKS-7 and NaSKS-11, as the alpha, beta and gamma forms. As noted above, the delta-Na₂SiO₅ (NaSKS-6 form) is most preferred for use herein. Other silicates may also be useful such as for example magnesium silicate, which can serve as a crisping agent in granular formulations, as a stabilizing agent for oxygen bleaches, and as a component of suds control systems.

Examples of carbonate builders are the alkaline earth and alkali metal carbonates as disclosed in German Patent Application No. 2,321,001 published on Nov. 15, 1973.

Conventional aluminosilicate builders may also be included. Aluminosilicate builders are of great importance in most currently marketed heavy duty granular detergent compositions, and can also be a significant builder ingredient in liquid detergent formulations. Aluminosilicate builders include those having the empirical formula:

M_{z}(zAlO₂)_{y}]·xH₂O

wherein z and y are integers of at least 6, the molar ratio of z to y is in the range from 1.0 to about 0.5, and x is an integer from about 15 to about 264.

Useful aluminosilicate ion exchange materials are commercially available. These aluminosilicates can be crystalline or amorphous in structure and can be naturally-occurring aluminosilicates or synthetically derived. A method for producing aluminosilicate ion exchange materials is disclosed in U.S. Pat. No. 3,985,669, Krummel, et al, issued Oct. 12, 1976. Preferred synthetic crystalline aluminosilicate ion exchange materials useful herein are available under the designations Zeolite A, Zeolite P (B), Zeolite MAP and Zeolite X. In an especially preferred embodiment, the crystalline aluminosilicate ion exchange material has the formula:

Na₁₂[(AlO₂)₁₂(SiO₂)₁₂]·xH₂O

wherein x is from about 20 to about 30, especially about 27. This material is known as Zeolite A. Dehydrated zeolites (x=0-10) may also be used herein. Preferably, the aluminosilicate has a particle size of about 1-7 microns in diameter.

Organic detergent builders suitable for the purposes of the present invention include, but are not restricted to, a wide variety of polycarboxylate compounds. As used herein, “polycarboxylate” refers to compounds having a plurality of carboxylate groups, preferably at least 3 carboxylates. Alkali metals, such as sodium, potassium, and lithium, or alkanolammonium salts are preferred.

Included among the polycarboxylate builders are a variety of categories of useful materials. One important category of polycarboxylate builders encompasses the ether polycarboxylates, as disclosed in Berg, U.S. Pat. No. 3,128,287, issued Apr. 7, 1964, and Lamberti et al, U.S. Pat. No. 3,635,830, issued Jan. 18, 1972. See also “TMS/TDS” builders of U.S. Pat. No. 4,663,071, issued to Bush et al, on May 5, 1987. Suitable ether polycarboxylates also include cyclic compounds, particularly alicyclic compounds, such as those described in U.S. Pat. Nos. 3,923,679; 3,835,163; 4,158,635; 4,120,874 and 4,102,903.

Citrate builders, e.g., citric acid salts (particularly sodium salt), are polycarboxylate builders of particular importance for liquid detergent formulations due to their availability from renewable resources and their biodegradability. Citrates can also be used in granular compositions, especially in combination with zeolite and/or layered silicate builders. Oxydisuccinates are also especially useful in such compositions and combinations.

Also suitable in the detergent compositions of the present invention are the 3,3-dicarboxy-4-oxa-1,6-hexanedioates and the related compounds disclosed in U.S. Pat. No. 4,566,984, Bush, issued Jan. 28, 1986. Useful succinic acid builders include the C₅-C₂₀ alkyl and alkenyl succinic acids salts. Specific examples of succinate builders include: laurylsuccinate, myristylsuccinate, palmitylsuccinate, 2-dodecenylsuccinate (preferred), 2-pentadecenylsuccinate, and the like. Laurylsuccinates are the preferred builders of this group, and are described in European Patent Application 86200690.5/0,200,263, published Nov. 5, 1986.

Other suitable polycarboxylates are disclosed in U.S. Pat. No. 4,144,226, Crutchfield et al, issued Mar. 13, 1979 and in U.S. Pat. No. 3,308,067, Diehl, issued Mar. 7, 1967. See also Diehl U.S. Pat. No. 3,723,322.

Monocarboxylic salts, can also be incorporated into the compositions alone. or in combination with the aforesaid builders, especially citrate and/or the succinate builders, to provide additional builder activity.

In situations where phosphorus-based builders can be used, the various alkali metal phosphates such as the well-known sodium tripolyphosphates, sodium pyrophosphate and sodium orthophosphate can be used. Phosphonate builders such as ethane-1-hydroxy-1,1-diphosphonate and other known phosphonates (see, for example, U.S. Pat. Nos. 3,159,581; 3,213,030; 3,422,021; 3,400,148 and 3,422,137) can also be used.

Adjunct Ingredients

The compositions herein can optionally include one or more other detergent adjunct materials or other materials for assisting or enhancing cleaning performance, treatment of the substrate to be cleaned, or to modify the aesthetics of the detergent composition (e.g., perfumes, colorants, dyes, etc.). The following are illustrative examples of such adjunct materials.

Enzymes—Enzymes can be included in the formulations herein for a wide variety of fabric laundering purposes, including removal of protein-based, carbohydrate-based, or triglyceride-based stains, for example, and for the prevention of refugee dye transfer, and for fabric restoration. The enzymes to be incorporated include proteases, amylases, lipases, cellulases, and peroxidases, as well as mixtures thereof. Other types of enzymes may also be included. They may be of any suitable origin, such as vegetable, animal, bacterial, fungal and yeast origin. However, their choice is governed by several factors such as pH-activity and/or stability optima, thermostability, stability versus active detergents, builders and so on. In this respect bacterial or fungal enzymes are preferred, such as bacterial amylases and proteases, and fungal cellulases.

Enzyme Stabilizers—The enzymes employed herein are stabilized by the presence of water-soluble sources of calcium and/or magnesium ions in the finished compositions which provide such ions to the enzymes. (Calcium ions are generally somewhat more effective than magnesium ions and are preferred herein if only one type of cation is being used.) Additional stability can be provided by the presence of various other art-disclosed stabilizers, especially borate species: see Severson, U.S. Pat. No. 4,537,706.

Bleaching Compounds—Bleaching Agents and Bleach Activators—The detergent compositions herein may optionally contain bleaching agents or bleaching compositions containing a bleaching agent and one or more bleach activators. When present, bleaching agents will typically be at levels of from about 1% to about 30%, more typically from about 5% to about 20%, of the detergent composition, especially for fabric laundering. If present, the amount of bleach activators will typically be from about 0.1% to about 60%, more typically from about 0.5% to about 40% of the bleaching composition comprising the bleaching agent-plus-bleach activator. Mixtures of bleaching agents can also be used. One preferred bleaching agent that is particularly useful wherein the pH is in the range of 8.5-9.5, in tablet applications is phthaloyl amido peroxy caproic acid (PAP).

Chelating Agents—The detergent compositions herein may also optionally contain one or more iron and/or manganese chelating agents. Such chelating agents can be selected from the group consisting of amino carboxylates, amino phosphonates, polyfunctionally-substituted aromatic chelating agents and mixtures therein, all as hereinafter defined. Without intending to be bound by theory, it is believed that the benefit of these materials is due in part to their exceptional ability to remove iron and manganese ions from washing solutions by formation of soluble chelates.

Polymeric Dispersing Agents—Polymeric dispersing agents can advantageously be utilized at levels from about 0.1% to about 7%, by weight, in the compositions herein, especially in the presence of zeolite and/or layered silicate builders. Suitable polymeric dispersing agents include polymeric polycarboxylates and polyethylene glycols, although others known in the art can also be used. It is believed, though it is not intended to be limited by theory, that polymeric dispersing agents enhance overall detergent builder performance, when used in combination with other builders (including lower molecular weight polycarboxylates) by crystal growth inhibition, particulate soil release peptization, and anti-redeposition.

Brightener—Any optical brighteners or other brightening or whitening agents known in the art can be incorporated at levels typically from about 0.05% to about 1.2%, by weight, into the detergent compositions herein. Commercial optical brighteners which may be useful in the present invention can be classified into subgroups, which include, but are not necessarily limited to, derivatives of stilbene, pyrazoline, coumarin, carboxylic acid, methinecyanines, dibenzothiophene-5,5-dioxide, azoles, 5- and 6-membered-ring heterocycles, and other miscellaneous agents. Examples of such brighteners are disclosed in “The Production and Application of Fluorescent Brightening Agents”, M. Zahradnik, Published by John Wiley & Sons, New York (1982).

Fabric Integrity Polymers—Linear amine based polymer, oligomer or copolymer materials which are suitable for use in laundry operations and provide the desired fabric appearance and integrity benefits can be characterized by the following general formula:

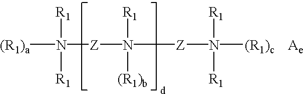

wherein:

each R₁ is independently selected from the group consisting of H, linear or branched C₁-C₁₂ alkyl, hydroxyalkyl, cycloalkyl, aryl, alkylaryl, piperidinoalkyl and other substituted derivatives of piperidine, morpholinoalkyl and other substituted derivatives of morpholine, substituted derivatives of aryl, substituted derivatives of alkylaryl,

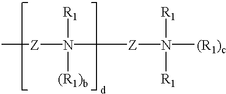

and mixtures thereof;

A is a compatible anion;

a=0 or 1;

b=0 or 1;

c=0 or 1;

d=from 0 to about 50, preferably from 0 to about 25 and most preferably from about 4 to about 20;

e=number required to obtain charge neutrality;

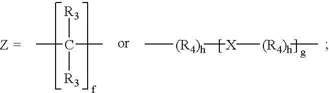

wherein:

each R₃ is independently selected from the group consisting of H, C₁-C₁₂ alkyl, aryl, alkylaryl, substituted derivatives of aryl, substituted derivatives of alkylaryl, hydroxy, amino, alkoxy, halogen and mixtures thereof;

each R₄ is independently selected from the group consisting of linear or branched alkylene, hydroxyalkylene, and substituted alkylene residues;

X is selected from the group consisting of phenylene, cyclohexylene, substituted residues of phenylene, substituted residues of cyclohexylene, —O—, —COO— and —CON(R₅)—;

R₅ is selected from the group consisting of H, C₁-C₄ alkyl and hydroxyalkyl;

f=from about 2 to about 12;

g=from about 1 to about 10 when X is —COO— or —CON(R₅)—;

g=from about 1 to about 100 when X is —O—; otherwise

g=1;

h=0 or 1;

provided that when one R₃ group is hydroxy or amino, the other R₃ group on the same carbon is not a hydroxy, amino or halogen; and

further provided that within Z no carbon has more than one substituent selected from the group consisting of hydroxy, amino, and halo.

The linear amine based polymer, oligomer or copolymer materials defined above can be used as a washing solution additive in either granular or liquid form. Alternatively, they can be admixed to granular detergents, or dissolved in liquid detergent compositions. The linear amine based fabric treatment component of the detergent compositions herein will generally comprise from about 0.1% to about 5% by the weight of the detergent composition. More preferably, such linear amine based fabric treatment materials will comprise from about 0.5% to about 4% by weight of the detergent compositions, most preferably from about 0.75% to about 3%.

Other Ingredients—A wide variety of other ingredients useful in detergent compositions can be included in the compositions herein, including other active ingredients, carriers, hydrotropes, processing aids, dyes or pigments, solvents for liquid formulations, solid fillers for bar compositions, etc. If desired, soluble magnesium salts such as MgCl₂, MgSO₄, and the like, can be added at levels of, typically, 0.1%-2%, to provide additional suds and to enhance grease removal performance.

Various detersive ingredients employed in the present compositions optionally can be further stabilized by absorbing said ingredients onto a porous hydrophobic substrate, then coating said substrate with a hydrophobic coating. Preferably, the detersive ingredient is admixed with a surfactant before being absorbed into the porous substrate. In use, the detersive ingredient is released from the substrate into the aqueous washing liquor, where it performs its intended detersive function.

The following examples are presented to further illustrate, but not to limit, the present invention:

Abbreviations used in Examples

| | |
|---|---|
| LAS | Sodium C₁₁₋₁₃ alkyl benzene sulfonate (linear, branched or |
| | mixed) |
| Alkyl Sulfate | CxyAS: Alkyl sulfate, typically sodium salt form, derived from |
| | fatty alcohol containing from x to y carbon atoms. Examples |
| | include sodium tallow alkyl sulfate (TAS) and primary, guerbet, |
| | and mid-chain branched alkyl sulfates containing from 10 to 20 |
| | carbon atoms (more typically from 14 to 16 or from 16 to 18) or |
| | mixtures thereof. |
| MBASₓ | Mid-chain branched primary alkyl (average total carbons = x) |
| | sulfate |
| MLAS | Crystallinity disrupted Sodium alkyl benzene sulfonate |
| Nonionic | linear or branched nonionic surfactant, typically CxyEz, derived |
| | from fatty alcohol with chainlength of from x to y condensed |
| | with an average of z moles of ethylene oxide Suitable examples |
| | include |
| | C25E3, C24E5 , C45E7. |
| Zeolite | Hydrated sodium aluminosilicate of formula |
| | Na₁₂(A10₂SiO₂)₁₂.27H₂O having an average particle size |
| | from 2 to 5 micrometers. |
| CMOS | Carboxymethyloxysuccinate and/or Salts |
| NTA | Nitrilotriacetic Acid and/or Salts |
| MGDA | Methyglycine Diacetic Acid and/or salts |
| Carbonate | Anhydrous sodium or potassium carbonate, e.g., with particle |
| | size between 200 μm and 900 μm for admix; or lower, e.g., |
| | below 100 μm, if to be further agglomerated. |
| NOBS | nonanoyloxybenzene sulfonate in the form of the sodium salt; |
| | NAC-OBS, i.e., (6-nonamidocaproyl) oxybenzene sulfonate; |
| | mixtures; or similar |
| Organic Bleach | e.g., omega-(3,4-dihydroisoquinolinium alkane sulfonate(s) of |
| Booster | |
| Inorganic Bleach | Dichioro manganese diethyl ethylene-bridged cyclam |
| Activator | |
| Photobleach | Sulfonated zinc phthlocyanine encapsulated in bleach dextrin |
| | soluble polymer; or low-hue photobleach. |
| PAP | Phthaloyl arnido peroxy caproic acid |
| Brightener | Disodium 4,4’-bis(2-sulphostyryl)biphenyl; Disodium 4,4′- |
| | bis(4-anilino-6-morpholino- 1.3.5-triazin-2-yl)amino) stilbene- |
| | 2:2′-disulfonate; mixtures |
| Antifoam System: | e.g., polydimethylsiloxane foam controller with siloxane- |
| | oxyalkylene copolymer as dispersing agent with a ratio of said |
| | foam controller to said dispersing agent of 10:1 to 100:1; may |
| | be complemented by fatty acid(s). |
| Tablet Coating | Adipic acid, Purolite mixture |

Examples

| | A | B | C | D | E | F | G | H |
|---|---|---|---|---|---|---|---|---|
| | Detergent | Detergent | Detergent | Detergent | Granular | Granular | Granular | Granular |
| | tablet | tablet | tablet | tablet | detergent | detergent | detergent | detergent |
| MBAS | 7.5 | 7.5 | 7.5 | 7.5 | 7.5 | 7.5 | 7.5 | 7.5 |
| C45AS | 5.25 | 5.25 | 5.25 | 5.25 | 5.25 | 5.25 | 5.25 | 5.25 |
| MLAS or LAS | 2.25 | 2.25 | 2.25 | 2.25 | | | 2.25 | |
| C23E6.5 | | | | | 2.25 | | | 2.25 |
| NTA Acid | 10 | 10 | 10 | | 10 | | | 10 |
| MGDA Acid | | | | | | | 8 | |
| CMOS Acid | | | | 10 | | | | |
| ODS Acid | | | | | | 10 | | |
| Zeolite | 20 | | 20 | 20 | 20 | 20 | 22 | |
| trisodium NTA | | 15 | | | | | | 15 |
| PAA | 2 | 2 | 2 | 2 | 2 | 2 | 2 | 2 |
| Sodium | 29.99 | 30 | 29.99 | 30 | 30 | 25 | 30 | 35 |
| carbonate | | | | | | | | |
| Percarbonate | 2 | 2 | 2 | | 2 | 2 | 2 | 2 |
| Protease | | | | | | | | |
| Enzyme | 0.3 | 0.3 | 0.3 | 0.3 | 0.3 | 0.3 | 0.3 | 0.3 |
| Brightener | 0.2 | 0.2 | 0.2 | 0.2 | 0.2 | 0.2 | 0.2 | 0.2 |
| PEG 4000 | 3 | 1 | 3 | 3 | 1 | 1 | 1 | 1 |
| Silicone | | | | | | | | |
| Antifoam | 0.2 | 0.2 | 0.2 | 0.2 | 0.2 | 0.2 | 0.2 | 0.2 |
| NaSO4 | 12.3 | 14.3 | 12.3 | 9.3 | 19.3 | 19.3 | 19.3 | 19.3 |
| Tablet Coating | 5 | | 5 | 5 | | | | |
| Acetate | | 10 | | | | | | |
| PAP | | | | 5 | | 7.25 | | |
| Inorganic | | | | | | | | |
| Bleach | | | | | | | | |
| Activator | 0.008 | | 0.008 | | | | | |
| Totals | 100 | 100 | 100 | 100 | 100 | 100 | 100 | 100 |

What is claimed is:

1. A detergent composition comprising:
(1) a detersive surfactant system;
(2) an effervescent system comprising:
(a) an acid source comprising an acid or acid-salt blend having the formula
ZO₂C—(CR₅R₆)ₓ—C—R₁—R₂—M—C—R₃—R₄—(CR₇R₈)_{y}—CO₂Z;
wherein x is 0 or 1, y is 0 or 1, 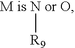
R₁, R₂, R₃, R₄, R₅, R₆, R₇, R₈ and R₉ are independently selected from the group consisting of H, OH, branched or linear alkyl hydrocarbon, branched or linear alkenyl hydrocarbon, branched or linear alcohol, branched or linear sulfonate or sulfate salt, branched or linear amine, branched or linear ether, branched or linear carboxylic acid, carboxylate salt substituents having a total of 6 or less total carbon units and mixtures thereof and wherein Z is selected from the group consisting of an alkali metal and hydrogen, with the proviso that on average greater than 1 Z, as measured in the complete acid or acid-salt blend, is hydrogen; and
(b) an alkali source;
wherein the acid source and the alkali source are capable of reacting together in the presence of water to form a gas; and
(c) optionally conventional detergent ingredients.

2. A detergent composition according to claim 1 wherein the alkali metal is selected from the group consisting of sodium and potassium.

3. A detergent composition according to claim 1 wherein the surfactant system comprises from about 0.01% to about 99% of the composition.

4. A detergent composition according to claim 1 wherein the effervescent system comprises from about 5% to about 50% by weight of the composition.

5. A detergent composition according to claim 1 wherein the effervescent system comprises from about 20% to about 80% by weight of the acid source and from about 80% to about 20% by weight of the alkali source.

6. A detergent composition according to claim 1 wherein x=0, y=0, M═N—R₉, R₁, R₂, R₃, and R₄ are all H and R₉ is CH₂CO₂Z.

7. A detergent composition according to claim 1 wherein x=0, y=0, M═N—R₉, R₁, R₂, R₃, and R₄ are all H and R₉ is CH(CH₃)CO₂Z.

8. A detergent composition according to claim 1 wherein x=1, y=1, M═N—R₉, R₁ and R₃ are H, R₂ and R₄ are CO₂Z, R₅, R₆, R₇, R₈ and R₉ are H.

9. A detergent composition according to claim 1 wherein x=1, y=1, M═N—R₉, R₁ and R₃ are H, R₂ and R₄ are CO₂Z, R₅, R₆, R₇, and R₉ are H and R₈ is OH.

11. A detergent composition according to claim 1 wherein x=0, y=1, M═O, R₁, R₂, R₃ , R₇ and R₈ are H, and R₄ is CO₂Z.

12. A detergent composition according to claim 1 wherein x=1, y=1, M═O, R₁R₃, R₅, R₆, R₇ and R₈ are H, R₂ and R₄ are CO₂Z.

13. A detergent composition according to claim 1 wherein the alkali source is sodium carbonate.

14. A detergent composition according to claim 1 wherein the alkali source is sodium bicarbonate.

15. A detergent composition according to claim 1 wherein the alkali source is potassium carbonate.

16. A detergent composition according to claim 1 wherein the alkali source is potassium bicarbonate.

17. A detergent composition according to claim 1 in the form of a tablet.
